(19) United States
(12) Patent Application Publication
(10) Pub. No.: US 2018/0169642 A1
(43) Pub. Date: Jun. 21, 2018

(54) CATALYST SYSTEM FOR ETHYLENE OLIGOMERIZATION AND METHOD FOR PRODUCING ETHYLENE OLIGOMERIZATION USING THE SAME

(71) Applicant: HANWHA TOTAL PETROCHEMICAL CO., LTD., Seosan-si (KR)

(72) Inventors: Chun Byung YANG, Seosan-si (KR); Joon Ryeo PARK, Seosan-si (KR)

(21) Appl. No.: 15/836,352

(22) Filed: Dec. 8, 2017

(30) Foreign Application Priority Data
Dec. 15, 2016 (KR) 10-2016-0171656

(51) Int. Cl. B01J 31/22; C07C 2/32; B01J 31/14

(52) U.S. Cl. CPC B01J 31/2213; C07C 2/32; C07C 2531/22; B01J 31/143; C07C 2531/14; B01J 31/2226

(57) ABSTRACT

The present disclosure relates to a catalyst system for ethylene oligomerization and a method for producing ethylene oligomerization using the same and more particularly, to a catalyst system for ethylene oligomerization including a transition metal or transition metal precursor with a new structure, a ligand with a backbone structure expressed by the following Chemical Formula 1 or Chemical Formula 2, and a co-catalyst for providing an ethylene oligomer and a method for producing ethylene oligomerization using the same. [Chemical Formula 1] R¹—OC(═O)—Y¹—C(═O)OR² Herein, R¹, R² are each independently hydrocarbyl, substituted hydrocarbyl, heterohydrocarbyl, or substituted heterohydrocarbyl, and Y¹ represents a group connecting CO(═O). [Chemical Formula 2] R¹—O—Y²—O—R² Herein, R¹, R² are each independently hydrocarbyl, substituted hydrocarbyl, heterohydrocarbyl, or substituted heterohydrocarbyl, and Y² represents a group connecting O and is a linear, branched, or cyclic alkyl group having 3 or more carbon atoms, or hetero hydrocarbyl, or substituted heterohydrocarbyl. The catalyst system of the present disclosure has an excellent catalytic activity and in the distribution of the produced α-olefins, C8″-C18″ α-olefins are highly distributed.

### CROSS-REFERENCE TO RELATED APPLICATION

This application claims the benefit and priority of Korean Patent Application No. 10-2016-0171656 filed Dec. 15, 2016. The entire disclosure of the above application is incorporated herein by reference.

### FIELD

The present disclosure relates to a catalyst system for use in olefin oligomerization reaction and a method for producing olefin oligomerization using the same and more particularly, to a catalyst for use in new oligomerization, a method for producing the same, and a method for producing ethylene oligomerization using the same.

### BACKGROUND

This section provides background information related to the present disclosure which is not necessarily prior art.

A conventional ethylene oligomerization technology is a catalyst technology for producing various α-olefins with Schulze-Flory or Poisson distribution and is also referred to as a full-range catalyst technology in the art. A catalyst technology for more selectively producing 1-butene, 1-hexene, or 1-octene is also referred to as an on-purpose technology. In recent years, a catalyst technology for more selectively producing 1-hexene or 1-octene has been greatly advanced.

The use of 1-butene, 1-hexene, or 1-octene has been greatly expanded to function as a co-monomer in production of linear low-density polyolefin. The use of the other various α-olefins having 10 or more carbon atoms is being expanded to serve as materials of detergent alcohols, lubricants for oil field, or wax, and the amount of the other α-olefins used is being greatly increased. The full-range catalyst technology has a long history, and a representative example thereof is a Ni-based catalyst being used in a SHOP processor developed by Shell. In this regard, EP0,177,999 and U.S. Pat. No. 3,676,523 illustrate a catalyst system from a diphenylphosphino acetic acid ligand and a Ni compound, and U.S. Pat. No. 4,528,416 illustrates a method of oligomerization of the catalyst in a mono-alcohol or diol solvent. Besides, DE1,443,927 and U.S. Pat. No. 3,906,053 illustrate a method of oligomerization of ethylene under a high ethylene pressure using a trialkyl aluminum catalyst. A method of oligomerization of ethylene with a catalyst system including zirconium alkoxide, alcohol, and an aluminum compound in the presence of solvents of toluene, cylcohexane, and normal-octane is illustrated in U.S. Pat. No. 6,930,218. However, the above-described catalysts have relatively low catalytic activity. EP0,444,505 discloses a processor for producing α-olefin using a Ziegler catalyst. The production of α-olefin is carried out efficiently but requires a relatively high ethylene pressure and a high temperature.

In recent years, the on-purpose catalyst technology of selectively trimerizing or tetramerizing ethylene into 1-hexene or 1-octene using various catalyst technologies has been greatly advanced, and most catalysts are based on chromium catalysts. As disclosed in U.S. Pat. No. 5,198,563, U.S. Pat. No. 5,376,612, and EP0,608,447, a high-activity and high-selectivity ethylene trimerization catalyst system commercialized by Phillips is based on a trivalent chromium compound, a pyrrole compound, and aluminum alkyl. In recent years, it has been disclosed that a chromium-based catalyst including a chelate ligand including hetero atoms of phosphorous and nitrogen selectively trimerizes or tetramerizes ethylene into 1-hexene or 1-octene (U.S. Pat. No. 7,964,763), and examples of the catalyst include (phenyl)₂PN(isopropyl)P(phenyl)₂. The above-described prior art technologies are limited to the selective production of mainly 1-hexene or 1-octene α-olefin with a chromium catalyst including a chelate ligand including hetero atoms and the chelate ligand is limited to a PNP backbone structure such as (R₁)(R₂)P—N(R₅)-P(R₃)(R₄). Also, a high-selectivity tetramerization catalyst system is disclosed in KR1,074,202 and based on a catalyst system including a chromium compound, a di-phosphine ligand with a PC CP backbone structure, and a co-catalyst compound.

As can be seen from the above description, the development of the full-range or on-purpose α-olefin production technology has been based on the advancement of various catalyst technologies, particularly a new ligand structure, and various requirements of α-olefins for development of various applications and uses need an improved catalyst.

### SUMMARY

This section provides a general summary of the disclosure, and is not a comprehensive disclosure of its full scope or all of its features.

The present disclosure has been made in an effort to provide a catalyst system for ethylene oligomerization and a method for producing ethylene oligomerization using the same and more particularly, a catalyst system for ethylene oligomerization including a transition metal or transition metal precursor with a new structure, a ligand with a backbone structure expressed by the following Chemical Formula 1 or Chemical Formula 2, and a co-catalyst for providing an ethylene oligomer and a method for producing ethylene oligomerization using the same.

R¹—OC(═O)—Y¹—C(═O)OR² [Chemical Formula 1]

Herein, R¹, R² are each independently hydrocarbyl, substituted hydrocarbyl, heterohydrocarbyl, or substituted heterohydrocarbyl, and Y¹ represents a group connecting CO(═O).

R¹—O—Y²—O—R² [Chemical Formula 2]

Herein, R¹, R² are each independently hydrocarbyl, substituted hydrocarbyl, heterohydrocarbyl, or substituted heterohydrocarbyl, and Y² represents a group connecting O and is a linear, branched, or cyclic alkyl group having 3 or more carbon atoms, or hetero hydrocarbyl, or substituted heterohydrocarbyl.

The catalyst system for ethylene oligomerization according to the present disclosure includes a transition metal or transition metal precursor, a ligand with a backbone structure of R¹—OC(═O)—Y¹—C(═O)OR² or R¹—O—Y²—O—R², and a co-catalyst, and the ligand with a backbone structure of R¹—OC(═O)—Y¹—C(═O)OR² or R¹—O—Y²—O—R² is expressed by the following Chemical Formula 1 or Chemical Formula 2.

R¹—OC(═O)—Y¹—C(═O)OR² [Chemical Formula 1]

Herein, R¹, R² are each independently hydrocarbyl, substituted hydrocarbyl, heterohydrocarbyl, or substituted heterohydrocarbyl, and Y¹ represents a group connecting CO(═O).

R¹—O—Y²—O—R² [Chemical Formula 2]

Herein, R¹, R² are each independently hydrocarbyl, substituted hydrocarbyl, heterohydrocarbyl, or substituted heterohydrocarbyl, and Y² represents a group connecting O and is a linear, branched, or cyclic alkyl group having 3 or more carbon atoms, or hetero hydrocarbyl, or substituted heterohydrocarbyl.

In the above description, R¹ and R² are each independently a hydrocarbyl group, a substituted hydrocarbyl group, or a substituted hetero hydrocarbyl group adjacent to O or C(═O)O, and these arbitrary substituents may be non-electron donors. These substituents may be nonpolar groups.

Preferably, R¹ and R² may be substituted aromatic groups or substituted heteroaromatic groups which do not include non-electron donors on atoms adjacent to the atom bonded to an O atom or C(═O)O group.

Preferred examples of R¹ and R² may be each independently selected from the group consisting of phenyl, benzyl, naphthyl, anthracenyl, mesityl, methyl, ethyl, propyl, isopropyl, butyl, isobutyl, tertiary butyl, cyclohexyl, 4-methylcyclohexyl, 4-ethylcyclohexyl, 4-isopropylcyclohexyl, tolyl, xylyl, 4-methylphenyl, 4-ethylphenyl, 4-isopropylphenyl, 4-t-butylphenyl, 4-methoxyphenyl, 4-isopropoxyphenyl, cumyl, methoxy, ethoxy, phenoxy, tolyloxy, dimethylamino, thiomethyl, trimethylsilyl, and dimethylhydrazyl. Preferably, R¹ and R² may be each independently selected from the group consisting of methyl, ethyl, propyl, isopropyl, butyl, isobutyl, tertiary butyl phenyl, tolyl, biphenyl, naphthyl, cyclohexyl, 4-methylphenyl, 4-ethylphenyl, 4-isopropylphenyl, 4-t-butylphenyl, 4-methoxyphenyl, and 4-isopropoxyphenyl.

R¹ and R² may be each independently an aromatic group and a substituted aromatic group, and each of R¹ and R² may be substituted with a non-electron donor group on at least one atom thereof, which is not adjacent to the atom bonded to an O atom or C(═O)O group.

Y¹ and Y² may be each independently a group connecting an O atom or C(═O)O group, and may be each independently a hydrocarbyl group, a substituted hydrocarbyl group, or a substituted heterohydrocarbyl group. These substituents may be nonpolar groups. Examples of Y¹ may include methylene, 1,2-ethane, 1,2-phenylene, 1,3-propane, 1,4-butane, 1,5-pentane, and the like, and examples of Y² may include 1,2-phenylene, 1,3-propane, 1,4-butane, 1,5-pentane, and the like.

Examples of the ligand with a backbone structure of R¹—OC(═O)—Y¹—C(═O)OR² or R¹—O—Y²—O—R² according to the present disclosure may include the following structure. However, the following structure example is provided only for illustrating the present disclosure, but does not limit the protective scope of Chemical Formula 1 of the present disclosure.

Representative structure examples of Chemical Formula 1 may include diether and dicarboxylic acid ester compounds. The diether compounds may include 1,3-diether-based compounds.

R¹R²C(CH₂OR³)(CH₂OR⁴) (1)

Herein, R¹ and R² are identical or different and represent C1-C18 alkyl groups, C3-C18 cycloalkyl groups, or C7-C18 aryl radical groups; and R³ and R⁴ are identical or different and represent C1-C4 alkyl radical groups or cyclic or polycyclic groups in which the carbon atom at position 2 contains 2 or 3 unsaturated bonds and which have 5, 6, or 7 carbon atoms.

Examples of the 1,3-diether-based compounds may include 2,2-diisobutyl-1,3-dimethoxypropane, 2-isopropyl-2-isobutyl-1,3-dimethoxypropane, 2-isopropyl-2-isopentyl-1,3-dimethoxypropane, 2,2-dicyclohexyl-1,3-dimethoxypropane, 9,9-bis(methoxymethyl)fluorene, and the like.

Further, the diether compounds may include cyclic polyene 1,3-diether. Examples of the cyclic polyene 1,3-diether may include 1,1-bis(methoxymethyl)-cyclopentadiene, 1,1-bis(methoxymethyl)-2,3,4,5-tetramethylcyclopentadiene, 1,1-bis(methoxymethyl)-2,3,4,5-tetraphenylcyclopentadiene, 1,1-bis(methoxymethyl)-2,3,4,5-tetrafluorocyclopentadiene, 1,1-bis(methoxymethyl)-3,4-dicyclopentylcyclopentadiene, 1,1-bis(methoxymethyl)indene, 1,1-bis(methoxymethyl)-2,3-dimethylindene, 1,1-bis(methoxymethyl)-4,5,6,7-tetrahydroindene, 1,1-bis(methoxymethyl)-2,3,6,7-tetrafluoroindene, 1,1-bis(methoxymethyl)-4,7-dimethylindene, 1,1-bis(methoxymethyl)-3,6-dimethylindene, 1,1-bis(methoxymethyl)-4-phenylindene, 1,1-bis(methoxymethyl)-4-phenyl-2-methylindene, 1,1-bis(methoxymethyl)-4-cyclohexylindene, 1,1-bis(methoxymethyl)-7-(3,3,3-trifluoropropyl)indene, 1,1-bis(methoxymethyl)-7-trimethylsilylindene, 1,1-bis(methoxymethyl)-7-trifluoromethylindene, 1,1-bis(methoxymethyl)-4,7-dimethyl-4,5,6,7-tetrahydroindene, 1,1-bis(methoxymethyl)-7-methylindene, 1,1-bis(methoxymethyl)-7-cyclopentylindene, 1,1-bis(methoxymethyl)-7-isopropylindene, 1,1-bis(methoxymethyl)-7-cyclohexylindene, 1,1-bis(methoxymethyl)-7-t-butylindene, 1,1-bis(methoxymethyl)-7-t-butyl-2-methylindene, 1,1-bis(methoxymethyl)-7-phenylindene, 1,1-bis(methoxymethyl)-2-phenylindene, 1,1-bis(methoxymethyl)-1H-benz[e]indene, 1,1-bis(methoxymethyl)-1H-2-methylbenz[e]indene, 9,9-bis(methoxymethyl)fluorene, 9,9-bis(methoxymethyl)-2,3,6,7-tetramethylfluorene, 9,9-bis(methoxymethyl)-2,3,4,5,6,7-hexafluorofluorene, 9,9-bis(methoxymethyl)-2,3-benzofluorene, 9,9-bis(methoxymethyl)-2,3,6,7-dibenzofluorene, 9,9-bis(methoxymethyl)-2,7-diisopropylfluorene, 9,9-bis(methoxymethyl)-1,8-dichlorofluorene, 9,9-bis(methoxymethyl)-2,7-dicyclopentylfluorene, 9,9-bis(methoxymethyl)-1,8-difluorofluorene, 9,9-bis(methoxymethyl)-1,2,3,4-tetrahydrofluorene, 9,9-bis(methoxymethyl)-1,2,3,4,5,6,7,8-octahydrofluorene, 9,9-bis(methoxymethyl)-4-t-butylfluorene, 1,1-bis(1′-butoxyethyl)-cyclopentadiene, 1,1-bis(1′-isopropoxy-n-propyl)cyclopentadiene, 1-methoxymethyl-1-(1′-methoxyethyl)-2,3,4,5-tetramethylcyclopentadiene, 1,1-bis(α-methoxybenzyl)indene, 1,1-bis(phenoxymethyl)indene, 1,1-bis(1′-methoxyethyl)-5,6-dichloroindene, 1,1-bis(phenoxymethyl)-3,6-dicyclohexylindene, 1-methoxymethyl-1-(1′-methoxyethyl)-7-t-butylindene, 1,1-bis[2-(2′-methoxypropyl)]-2-methylindene, 3,3-bis(methoxymethyl)-3H-2-methylbenz[e]indene, 9,9-bis(α-methoxybenzyl)fluorene, 9,9-bis(1′-isopropoxy-n-butyl)-4,5-diphenylfluorene, 9,9-bis(1′-methoxyethyl)fluorene, 9-(methoxymethyl)-9-(1′-methoxyethyl)-2,3,6,7-tetrafluorofluorene, 9-methoxymethyl-9-penthoxymethylfluorene; 9-methoxymethyl-9-ethoxymethylfluorene, 9-methoxymethyl-9-(1′-methoxyethyl)-fluorene, 9-methoxymethyl-9-[2-(2-methoxypropyl)]-fluorene, 1,1-bis(methoxymethyl)-2,5-cyclohexadiene, 1,1-bis(methoxymethyl)benzonaphthene, 7,7-bis(methoxymethyl)-2,5-norbornanediene, 9,9-bis(methoxymethyl)-1,4-methane dihydronaphthalene, 4,4-bis(methoxymethyl)-4H-cyclopenta[d, e, f]phenanthrene, 9,9-bis(methoxymethyl)-9,10-dihydroanthracene, 7,7-bis(methoxymethyl)-7H-benz[d,e]anthracene, 1,1-bis(methoxymethyl)-1,2-dihydronaphthalene, 4,4-bis(methoxymethyl)-1-phenyl-3,4-dihydronaphthalene, 4,4-bis(methoxymethyl)-1-phenyl-1,4-dihydronaphthalene, 5,5-bis(methoxymethyl)-1,3,6-cycloheptatriene, 5,5-bis(methoxymethyl)-10,11-dihydro-5H-dibenzo[a, d]cycloheptene, 5,5-bis(methoxymethyl)-5H-dibenzo[a,d]cycloheptene, 9,9-bis(methoxymethyl)xanthene, 9,9-bis(methoxymethyl)-2,3,6,7-tetramethylxanthene, 9,9-bis(1′-methoxyisobutyl)thioxanthene, 4,4-bis(methoxymethyl)-1,4-pyrane, 9,9-bis(methoxymethyl)-N-t-butyl-9,10-dihydroacrydine, 4,4-bis(methoxymethyl)-1,4-chromene, 4,4-bis(methoxymethyl)-1,2,4-oxazine, 1,1-bis(methoxymethyl)benzo-2,3,1-oxazine, 5,5-bis(methoxymethyl)-1,5-pyridine, 5,5-bis(methoxymethyl)-6,7-dimethyl-1,5-pyridine, 2,2-bis(methoxymethyl)-3,4,5-trifluoroisopyrrole, 4,4-bis(1′-methoxyethyl)benzo-N-phenyl-1,4-dihydropyridine, and the like.

The dicarboxylic acid ester compounds may have various structures. One example is a benzene-1,2-dicarboxylic acid ester compound.

Specific examples of the benzene-1,2-dicarboxylic acid ester compound may include dimethylphthalate, diethylphthalate, dinormalpropylphthalate, diisopropylphthalate, dinormalbutylphthalate, diisobutylphthalate, dinormalpentylphthalate, di(2-methylbutyl)phthalate, di(3-methylbutyl)phthalate, dineopentylphthalate, dinormalhexylphthalate, di(2-methylpentyl)phthalate, di(3-methylpentyl)phthalate, diisohexylphthalate, dineohexylphthalate, di(2,3-dimethylbutyl)phthalate, dinormalheptylphthalate, di(2-methylhexyl)phthalate, di(2-ethylpentyl)phthalate, diisoheptylphthalate, dineoheptylphthalate, dinormaloctylphthalate, di(2-methylheptyl)phthalate, diisooctylphthalate, di(3-ethylhexyl)phthalate, dineooctylphthalate, dinormalnonylphthalate, diisononylphthalate, dinormaldecylphthalate, diisodecylphthalate, and the like.

Further, the dicarboxylic acid ester may include malonate, succinate, glutarate, pivalate, adipate, sebacate, malate, naphthalene dicarboxylate, trimellitate, benzene-1,2,3-tricarboxylate, pyromellitate, and carbonate. Examples thereof may include diethyl malonate, dibutyl malonate, dimethylsuccinate, diethylsuccinate, dinormalpropyl succinate, diisopropylsuccinate, 1,1-dimethyl-dimethylsuccinate, 1,1-dimethyl-diethylsuccinate, 1,1-dimethyl-dinormalpropylsuccinate, 1,1-dimethyl-diisopropylsuccinate, 1,2-dimethyl-dimethylsuccinate, 1,2-dimethyl-diethylsuccinate, ethyl-dimethylsuccinate, ethyl-diethylsuccinate, ethyl-dinormalpropylsuccinate, ethyl-diisopropylsuccinate, 1,1-diethyl-dimethylsuccinate, 1,1-diethyl-diethylsuccinate, 1,1-diethyl-dimethylsuccinate, 1,2-diethyl-dimethylsuccinate, 1,2-diethyl-diethylsuccinate, 1,2-diethyl-dinormalpropylsuccinate, 1,2-diethyl-diisopropylsuccinate, normalpropyl-dimethylsuccinate, normalpropyl-diethylsuccinate, normalpropyl-dinormalpropylsuccinate, normalpropyl-diisopropylsuccinate, isopropyl-dimethylsuccinate, isopropyl-diethylsuccinate, isopropyl-dinormalpropylsuccinate, isopropyl-diisopropylsuccinate, 1,2-diisopropyl-dimethylsuccinate, 1,2-diisopropyl-diethylsuccinate, 1,2-diisopropyl-dinormalpropylsuccinate, 1,2-diisopropyl-diisopropylsuccinate, normalbutyl-dimethylsuccinate, normalbutyl-diethylsuccinate, normalbutyl-dinormalpropylsuccinate, normalbutyl-diisopropylsuccinate, isobutyl-dimethyl succinate, isobutyl-diethylsuccinate, isobutyl-dinormalpropylsuccinate, isobutyl-diisopropylsuccinate, 1,2-dinormalbutyl-dimethylsuccinate, 1,2-dinormalbutyl-diethylsuccinate, 1,2-dinormalbutyl-dinormalpropylsuccinate, 1,2-dinormalbutyl-diisopropylsuccinate, 1,2-dinormalbutyl-dimethylsuccinate, 1,2-diisobutyl-dimethylsuccinate, 1,2-diisobutyl-diethylsuccinate, 1,2-diisobutyl-dinormalpropylsuccinate, 1,2-diisobutyl-diisopropylsuccinate, diethyl adipate, dibutyl adipate, diethyl sebacate, dibutyl sebacate, diethyl malate, di-n-butyl malate, diethyl naphthalene dicarboxylate, dibutyl naphthalene dicarboxylate, triethyl trimellitate, tributyl trimellitate, triethyl benzene-1,2,3-tricarboxylate, tributyl benzene-1,2,3-tricarboxylate, tetraethyl pyromellitate, tetrabutyl pyromellitate, and the like.

The dicarboxylic acid ester compound may include General Formula 2 having the following structure.

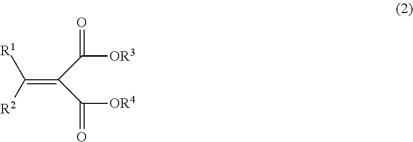

Herein, R¹ and R² are each independently hydrogen or a linear or branched alkyl group having 1 to 20 carbon atoms, a cyclic alkyl group or alkenyl group having 3 to 20 carbon atoms, an aryl group having 6 to 20 carbon atoms, or an arylalkyl group or alkylaryl group having 7 to 20 carbon atoms and are combined to form a cycle, and R³ and R⁴ are each independently a linear or branched alkyl group having 1 to 20 carbon atoms. Examples thereof may include diethyl 2-(1H-indene-2(3H)-ylidene)malonate, diethyl 2-(9H-fluorene-9-ylidene)malonate, diethyl 2-cyclobutylidene malonate, diethyl 2-cyclopentylidene malonate, diethyl 2-cyclohexylidene malonate, diethyl 2-methylene malonate, diethyl 2-ethylidene malonate, diethyl 2-propylidene malonate, diethyl 2-(2-methylpropylidene)malonate, diethyl 2-(2,2-dimethylpropylidene)malonate, diethyl 2-(cyclobutylmethylene)malonate, diethyl 2-(cyclopentylmethylene)malonate, diethyl 2-(cyclohexylmethylene)malonate, diethyl 2-(butane-2-ylidene)malonate, diethyl 2-(3-methylbutane-2-ylidene)malonate, diethyl 2-(3,3-dimethylbutane-2-ylidene)malonate, diethyl 2-(1-cyclobutylethylidene)malonate, diethyl 2-(1-cyclopentylethylidene)malonate, diethyl 2-(1-cyclohexylethylidene)malonate, diethyl 2-(2,4-dimethylpentane-3-ylidene)malonate, diethyl 2-(2,2,4,4,-tetramethylpentane-3-ylidene)malonate, diethyl 2-(dicyclobutylmethylene)malonate, diethyl 2-(dicyclopentylmethylene)malonate, diethyl 2-(dicyclohexylmethylene)malonate, dipropyl 2-(1H-indene-2 (3H)-ylidene)malonate, dipropyl 2-(9H-fluorene-9-ylidene)malonate, dipropyl 2-cyclobutylidene malonate, dipropyl 2-cyclopentylidene malonate, dipropyl 2-cyclohexylidene malonate, dipropyl 2-methylene malonate, dipropyl 2-ethylidene malonate, dipropyl 2-propylidene malonate, dipropyl 2-(2-methylpropylidene)malonate, dipropyl 2-(2,2-dimethylpropylidene)malonate, dipropyl 2-(cyclobutylmethylene)malonate, dipropyl 2-(cyclopentylmethylene)malonate, dipropyl 2-(cyclohexylmethylene)malonate, dipropyl 2-(butane-2-ylidene)malonate, dipropyl 2-(3-methylbutane-2-ylidene)malonate, dipropyl 2-(3,3-dimethylbutane-2-ylidene)malonate, dipropyl 2-(1-cyclobutylethylidene)malonate, dipropyl 2-(1-cyclopentylethylidene)malonate, dipropyl 2-(1-cyclohexylethylidene)malonate, dipropyl 2-(2,4-dimethylpentane-3-ylidene)malonate, dipropyl 2-(2,2,4,4,-tetramethylpentane-3-ylidene)malonate, dipropyl 2-(dicyclobutylmethylene)malonate, dipropyl 2-(dicyclopentylmethylene)malonate, dipropyl 2-(dicyclohexylmethylene)malonate, diisopropyl 2-(1H-indene-2(3H)-ylidene)malonate, diisopropyl 2-(9H-fluorene-9-ylidene)malonate, diisopropyl 2-cyclobutylidene malonate, diisopropyl 2-cyclopentylidene malonate, diisopropyl 2-cyclohexylidene malonate, diisopropyl 2-methylene malonate, diisopropyl 2-ethylidene malonate, diisopropyl 2-propylidene malonate, diisopropyl 2-(2-methylpropylidene)malonate, diisopropyl 2-(2,2-dimethylpropylidene)malonate, diisopropyl 2-(cyclobutylmethylene)malonate, diisopropyl 2-(cyclopentylmethylene) malonate, diisopropyl 2-(cyclohexylmethylene) malonate, diisopropyl 2-(butane-2-ylidene)malonate, diisopropyl2-(3-methylbutane-2-ylidene)malonate, diisopropyl 2-(3,3-dimethylbutane-2-ylidene)malonate, diisopropyl 2-(1-cyclobutylethylidene) malonate, diisopropyl 2-(1-cyclopentylethylidene)malonate, diisopropyl 2-(1-cyclohexylethylidene) malonate, diisopropyl 2-(2,4-dimethylpentane-3-ylidene) malonate, diisopropyl 2-(2,2,4,4,-tetramethylpentane-3-ylidene) malonate, diisopropyl 2-(dicyclobutylmethylene) malonate, diisopropyl 2-(dicyclopentylmethylene)malonate, diisopropyl 2-(dicyclohexylmethylene)malonate, dibutyl 2-(1H-indene-2(3H)-ylidene)malonate, dibutyl 2-(9H-fluorene-9-ylidene)malonate dibutyl 2-cyclobutylidene malonate, dibutyl 2-cyclopentylidene malonate, dibutyl 2-cyclohexylidene malonate, dibutyl 2-methylene malonate, dibutyl 2-ethylidene malonate, dibutyl 2-propylidene malonate, dibutyl 2-(2-methylpropylidene)malonate, dibutyl 2-(2,2-dimethylpropylidene)malonate, dibutyl 2-(cyclobutylmethylene)malonate, dibutyl 2-(cyclopentylmethylene)malonate, dibutyl 2-(cyclohexylmethylene)malonate, dibutyl 2-(butane-2-ylidene)malonate, dibutyl 2-(3-methylbutane-2-ylidene)malonate, dibutyl 2-(3,3-dimethylbutane-2-ylidene)malonate, dibutyl 2-(1-cyclobutylethylidene)malonate, dibutyl 2-(1-cyclopentylethylidene)malonate, dibutyl 2-(1-cyclohexylethylidene)malonate, dibutyl 2-(2,4-dimethylpentane-3-ylidene)malonate, dibutyl 2-(2,2,4,4,-tetramethylpentane-3-ylidene)malonate, dibutyl 2-(dicyclobutylmethylene)malonate, dibutyl 2-(dicyclopentylmethylene)malonate, dibutyl 2-(dicyclohexylmethylene)malonate, diisobutyl 2-(1H-indene-2(3H)-ylidene)malonate, diisobutyl 2-(9H-fluorene-9-ylidene)malonate, diisobutyl 2-cyclobutylidene malonate, diisobutyl 2-cyclopentylidene malonate, diisobutyl 2-cyclohexylidene malonate, diisobutyl 2-methylene malonate, diisobutyl 2-ethylidene malonate, diisobutyl 2-propylidene malonate, diisobutyl 2-(2-methylpropylidene)malonate, diisobutyl 2-(2,2-dimethylpropylidene)malonate, diisobutyl 2-(cyclobutylmethylene)malonate, diisobutyl 2-(cyclopentylmethylene)malonate, diisobutyl 2-(cyclohexylmethylene)malonate, diisobutyl 2-(butane-2-ylidene)malonate, diisobutyl 2-(3-methylbutane-2-ylidene)malonate, diisobutyl 2-(3,3-dimethylbutane-2-ylidene)malonate, diisobutyl 2-(1-cyclobutylethylidene)malonate, diisobutyl 2-(1-cyclopentylethylidene)malonate, diisobutyl 2-(1-cyclohexylethylidene)malonate, diisobutyl 2-(2,4-dimethylpentane-3-ylidene)malonate, diisobutyl 2-(2,2,4,4,-tetramethylpentane-3-ylidene)malonate, diisobutyl 2-(dicyclobutylmethylene)malonate, diisobutyl 2-(dicyclopentylmethylene)malonate, diisobutyl 2-(dicyclohexylmethylene)malonate, and the like.

The dicarboxylic acid ester compound may include a bicycloalkanedicarboxylate-based or bicycloalkenedicarboxylate-based compound represented by General Formula 3, General Formula 4, General Formula 5, or General Formula 6 having the following structure.

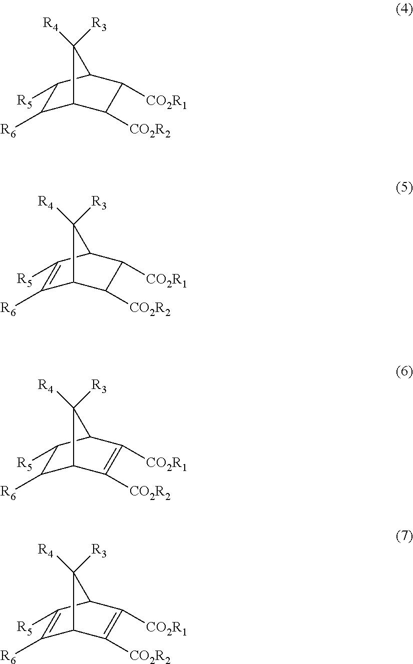

Herein, R₁ and R₂ are identical to or different from each other and represent linear, branched, or cyclic alkyl groups or alkenyl groups having 1 to 20 carbon atoms, aryl groups having 6 to 20 carbon atoms, or arylalkyl groups or alkylaryl groups having 7 to 20 carbon atoms; and R₃, R₄, R₅ and R₆ are identical to or different from each other and represent hydrogen, linear, branched, or cyclic alkyl groups or alkenyl groups having 1 to 20 carbon atoms, aryl groups having 6 to 20 carbon atoms, or arylalkyl groups or alkylaryl groups having 7 to 20 carbon atoms.

Examples of the bicycloalkanedicarboxylate-based or bicycloalkenedicarboxylate-based compound represented by General Formula 3, General Formula 4, General Formula 5, or General Formula 6 may include bicyclo[2.2.1]heptane-2,3-dicarboxylic acid ethylhexylester, bicyclo[2.2.1]heptane-2,3-dicarboxylic acid dioctylester, bicyclo[2.2.1]heptane-2,3-dicarboxylic acid diisobutylester, bicyclo[2.2.1]heptane-2,3-dicarboxylic acid dibutylester, bicyclo[2.2.1]heptane-2,3-dicarboxylic acid diisopropylester, bicyclo[2.2.1]heptane-2,3-dicarboxylic acid dipropylester, bicyclo[2.2.1]heptane-2,3-dicarboxylic acid diethylester, bicyclo[2.2.1]heptane-2,3-dicarboxylic acid dimethylester, 7,7-dimethylbicyclo[2.2.1]heptane-2,3-dicarboxylic acid ethylhexylester, 7,7-dimethylbicyclo[2.2.1]heptane-2,3-dicarboxylic acid dioctylester, 7,7-dimethylbicyclo[2.2.1]heptane-2,3-dicarboxylic acid diisobutylester, 7,7-dimethylbicyclo[2.2.1]heptane-2,3-dicarboxylic acid dibutylester, 7,7-dimethylbicyclo[2.2.1]heptane-2,3-dicarboxylic acid diisopropylester, 7,7-dimethylbicyclo[2.2.1]heptane-2,3-dicarboxylic acid dipropylester, 7,7-dimethylbicyclo[2.2.1]heptane-2,3-dicarboxylic acid diethylester, 7,7-dimethylbicyclo[2.2.1]heptane-2,3-dicarboxylic acid dimethylester, 5-methylbicyclo[2.2.1]heptane-2,3-dicarboxylic acid ethylhexylester, 5-methylbicyclo[2.2.1]heptane-2,3-dicarboxylic acid dioctylester, 5-methylbicyclo[2.2.1]heptane-2,3-dicarboxylic acid diisobutylester, 5-methylbicyclo[2.2.1]heptane-2,3-dicarboxylic acid dibutylester, 5-methylbicyclo[2.2.1]heptane-2,3-dicarboxylic acid diisopropylester, 5-methylbicyclo[2.2.1]heptane-2,3-dicarboxylic acid dipropylester, 5-methylbicyclo[2.2.1]heptane-2,3-dicarboxylic acid diethylester, 5-methylbicyclo[2.2.1]heptane-2,3-dicarboxylic acid dimethylester, 6-methylbicyclo[2.2.1]heptane-2,3-dicarboxylic acid ethylhexylester, 6-methylbicyclo[2.2.1]heptane-2,3-dicarboxylic acid dioctylester, 6-methylbicyclo[2.2.1]heptane-2,3-dicarboxylic acid diisobutylester, 6-methylbicyclo[2.2.1]heptane-2,3-dicarboxylic acid dibutylester, 6-methylbicyclo[2.2.1]heptane-2,3-dicarboxylic acid diisopropylester, 6-methylbicyclo[2.2.1]heptane-2,3-dicarboxylic acid dipropylester, 6-methylbicyclo[2.2.1]heptane-2,3-dicarboxylic acid diethylester, 6-methylbicyclo[2.2.1]heptane-2,3-dicarboxylic acid dimethylester, 5,6-dimethylbicyclo[2.2.1]heptane-2,3-dicarboxylic acid ethylhexylester, 5,6-dimethylbicyclo[2.2.1]heptane-2,3-dicarboxylic acid dioctylester, 5,6-dimethylbicyclo[2.2.1]heptane-2,3-dicarboxylic acid diisobutylester, 5,6-dimethylbicyclo[2.2.1]heptane-2,3-dicarboxylic acid dibutylester, 5,6-dimethylbicyclo[2.2.1]heptane-2,3-dicarboxylic acid diisopropylester, 5,6-dimethylbicyclo[2.2.1]heptane-2,3-dicarboxylic acid dipropylester, 5,6-dimethylbicyclo[2.2.1]heptane-2,3-dicarboxylic acid diethylester, 5,6-dimethylbicyclo[2.2.1]heptane-2,3-dicarboxylic acid dimethylester, bicyclo[2.2.1]hept-5-ene-2,3-dicarboxylic acid ethylhexylester, bicyclo[2.2.1]hept-5-ene-2,3-dicarboxylic acid dioctylester, bicyclo[2.2.1]hept-5-ene-2,3-dicarboxylic acid diisobutylester, bicyclo[2.2.1]hept-5-ene-2,3-dicarboxylic acid dibutylester, bicyclo[2.2.1]hept-5-ene-2,3-dicarboxylic acid diisopropylester, bicyclo[2.2.1]hept-5-ene-2,3-dicarboxylic acid dipropylester, bicyclo[2.2.1]hept-5-ene-2,3-dicarboxylic acid diethylester, bicyclo[2.2.1]hept-5-ene-2,3-dicarboxylic acid dimethylester, 7,7-dimethylbicyclo[2.2.1]hept-5-ene-2,3-dicarboxylic acid ethylhexylester, 7,7-dimethylbicyclo[2.2.1]hept-5-ene-2,3-dicarboxylic acid dioctylester, 7,7-dimethylbicyclo[2.2.1]hept-5-ene-2,3-dicarboxylic acid diisobutylester, 7,7-dimethylbicyclo[2.2.1]hept-5-ene-2,3-dicarboxylic acid dibutylester, 7,7-dimethylbicyclo[2.2.1]hept-5-ene-2,3-dicarboxylic acid diisopropylester, 7,7-dimethylbicyclo[2.2.1]hept-5-ene-2,3-dicarboxylic acid dipropylester, 7,7-dimethylbicyclo[2.2.1]hept-5-ene-2,3-dicarboxylic acid diethylester, 7,7-dimethylbicyclo[2.2.1]hept-5-ene-2,3-dicarboxylic acid dimethylester, 5-methylbicyclo[2.2.1]hept-5-ene-2,3-dicarboxylic acid ethylhexylester, 5-methylbicyclo[2.2.1]hept-5-ene-2,3-dicarboxylic acid dioctylester, 5-methylbicyclo[2.2.1]hept-5-ene-2,3-dicarboxylic acid diisobutylester, 5-methylbicyclo[2.2.1]hept-5-ene-2,3-dicarboxylic acid dibutylester, 5-methylbicyclo[2.2.1]hept-5-ene-2,3-dicarboxylic acid diisopropylester, 5-methylbicyclo[2.2.1]hept-5-ene-2,3-dicarboxylic acid dipropylester, 5-methylbicyclo[2.2.1]hept-5-ene-2,3-dicarboxylic acid diethylester, 5-methylbicyclo[2.2.1]hept-5-ene-2,3-dicarboxylic acid dimethylester, 6-methylbicyclo[2.2.1]hept-5-ene-2,3-dicarboxylic acid ethylhexylester, 6-methylbicyclo[2.2.1]hept-5-ene-2,3-dicarboxylic acid dioctylester, 6-methylbicyclo[2.2.1]hept-5-ene-2,3-dicarboxylic acid diisobutylester, 6-methylbicyclo[2.2.1]hept-5-ene-2,3-dicarboxylic acid dibutylester, 6-methylbicyclo[2.2.1]hept-5-ene-2,3-dicarboxylic acid diisopropylester, 6-methylbicyclo[2.2.1]hept-5-ene-2,3-dicarboxylic acid dipropylester, 6-methylbicyclo[2.2.1]hept-5-ene-2,3-dicarboxylic acid diethylester, 6-methylbicyclo[2.2.1]hept-5-ene-2,3-dicarboxylic acid dimethylester, 5,6-dimethylbicyclo[2.2.1]hept-5-ene-2,3-dicarboxylic acid ethylhexylester, 5,6-dimethylbicyclo[2.2.1]hept-5-ene-2,3-dicarboxylic acid dioctylester, 5,6-dimethylbicyclo[2.2.1]hept-5-ene-2,3-dicarboxylic acid diisobutylester, 5,6-dimethylbicyclo[2.2.1]hept-5-ene-2,3-dicarboxylic acid dibutylester, 5,6-dimethylbicyclo[2.2.1]hept-5-ene-2,3-dicarboxylic acid diisopropylester, 5,6-dimethylbicyclo[2.2.1]hept-5-ene-2,3-dicarboxylic acid dipropylester, 5,6-dimethylbicyclo[2.2.1]hept-5-ene-2,3-dicarboxylic acid diethylester, 5,6-dimethylbicyclo[2.2.1]hept-5-ene-2,3-dicarboxylic acid dimethylester, bicyclo[2.2.1]hept-2-ene-2,3-dicarboxylic acid ethylhexylester, bicyclo[2.2.1]hept-2-ene-2,3-dicarboxylic acid dioctylester, bicyclo[2.2.1]hept-2-ene-2,3-dicarboxylic acid diisobutylester, bicyclo[2.2.1]hept-2-ene-2,3-dicarboxylic acid dibutylester, bicyclo[2.2.1]hept-2-ene-2,3-dicarboxylic acid diisopropylester, bicyclo[2.2.1]hept-2-ene-2,3-dicarboxylic acid dipropylester, bicyclo[2.2.1]hept-2-ene-2,3-dicarboxylic acid diethylester, bicyclo[2.2.1]hept-2-ene-2,3-dicarboxylic acid dimethylester, 7,7-dimethylbicyclo[2.2.1]hept-2-ene-2,3-dicarboxylic acid ethylhexylester, 7,7-dimethylbicyclo[2.2.1]hept-2-ene-2,3-dicarboxylic acid dioctylester, 7,7-dimethylbicyclo[2.2.1]hept-2-ene-2,3-dicarboxylic acid diisobutylester, 7,7-dimethylbicyclo[2.2.1]hept-2-ene-2,3-dicarboxylic acid dibutylester, 7,7-dimethylbicyclo[2.2.1]hept-2-ene-2,3-dicarboxylic acid diisopropylester, 7,7-dimethylbicyclo[2.2.1]hept-2-ene-2,3-dicarboxylic acid dipropylester, 7,7-dimethylbicyclo[2.2.1]hept-2-ene-2,3-dicarboxylic acid diethylester, 7,7-dimethylbicyclo[2.2.1]hept-2-ene-2,3-dicarboxylic acid dimethylester, 5-methylbicyclo[2.2.1]hept-2-ene-2,3-dicarboxylic acid ethylhexylester, 5-methylbicyclo[2.2.1]hept-2-ene-2,3-dicarboxylic acid dioctylester, 5-methylbicyclo[2.2.1]hept-2-ene-2,3-dicarboxylic acid diisobutylester, 5-methylbicyclo[2.2.1]hept-2-ene-2,3-dicarboxylic acid dibutylester, 5-methylbicyclo[2.2.1]hept-2-ene-2,3-dicarboxylic acid diisopropylester, 5-methylbicyclo[2.2.1]hept-2-ene-2,3-dicarboxylic acid dipropylester, 5-methylbicyclo[2.2.1]hept-2-ene-2,3-dicarboxylic acid diethylester, 5-methylbicyclo[2.2.1]hept-2-ene-2,3-dicarboxylic acid dimethylester, 6-methylbicyclo[2.2.1]hept-2-ene-2,3-dicarboxylic acid ethylhexylester, 6-methylbicyclo[2.2.1]hept-2-ene-2,3-dicarboxylic acid dioctylester, 6-methylbicyclo[2.2.1]hept-2-ene-2,3-dicarboxylic acid diisobutylester, 6-methylbicyclo[2.2.1]hept-2-ene-2,3-dicarboxylic acid dibutylester, 6-methylbicyclo[2.2.1]hept-2-ene-2,3-dicarboxylic acid diisopropylester, 6-methylbicyclo[2.2.1]hept-2-ene-2,3-dicarboxylic acid dipropylester, 6-methylbicyclo[2.2.1]hept-2-ene-2,3-dicarboxylic acid diethylester, 6-methylbicyclo[2.2.1]hept-2-ene-2,3-dicarboxylic acid dimethylester, 5,6-dimethylbicyclo[2.2.1]hept-2-ene-2,3-dicarboxylic acid ethylhexylester, 5,6-dimethylbicyclo[2.2.1]hept-2-ene-2,3-dicarboxylic acid dioctylester, 5,6-dimethylbicyclo[2.2.1]hept-2-ene-2,3-dicarboxylic acid diisobutylester, 5,6-dimethylbicyclo[2.2.1]hept-2-ene-2,3-dicarboxylic acid dibutylester, 5,6-dimethylbicyclo[2.2.1]hept-2-ene-2,3-dicarboxylic acid diisopropylester, 5,6-dimethylbicyclo[2.2.1]hept-2-ene-2,3-dicarboxylic acid dipropylester, 5,6-dimethylbicyclo[2.2.1]hept-2-ene-2,3-dicarboxylic acid diethylester, 5,6-dimethylbicyclo[2.2.1]hept-2-ene-2,3-dicarboxylic acid dimethylester, bicyclo[2.2.1]hept-2,5-dien-2,3-dicarboxylic acid ethylhexylester, bicyclo[2.2.1]hept-2,5-dien-2,3-dicarboxylic acid dioctylester, bicyclo[2.2.1]hept-2,5-dien-2,3-dicarboxylic acid diisobutylester, bicyclo[2.2.1]hept-2,5-dien-2,3-dicarboxylic acid dibutylester, bicyclo[2.2.1]hept-2,5-dien-2,3-dicarboxylic acid diisopropylester, bicyclo[2.2.1]hept-2,5-dien-2,3-dicarboxylic acid dipropylester, bicyclo[2.2.1]hept-2,5-dien-2,3-dicarboxylic acid diethylester, bicyclo[2.2.1]hept-2,5-dien-2,3-dicarboxylic acid dimethylester, 7,7-dimethylbicyclo[2.2.1]hept-2,5-dien-2,3-dicarboxylic acid ethylhexylester, 7,7-dimethylbicyclo[2.2.1]hept-2,5-dien-2,3-dicarboxylic acid dioctylester, 7,7-dimethylbicyclo[2.2.1]hept-2,5-dien-2,3-dicarboxylic acid diisobutylester, 7,7-dimethylbicyclo[2.2.1]hept-2,5-dien-2,3-dicarboxylic acid dibutylester, 7,7-dimethylbicyclo[2.2.1]hept-2,5-dien-2,3-dicarboxylic acid diisopropylester, 7,7-dimethylbicyclo[2.2.1]hept-2,5-dien-2,3-dicarboxylic acid dipropylester, 7,7-dimethylbicyclo[2.2.1]hept-2,5-dien-2,3-dicarboxylic acid diethylester, 7,7-dimethylbicyclo[2.2.1]hept-2,5-dien-2,3-dicarboxylic acid dimethylester, 5-methylbicyclo[2.2.1]hept-2,5-dien-2,3-dicarboxylic acid ethylhexylester, 5-methylbicyclo[2.2.1]hept-2,5-dien-2,3-dicarboxylic acid dioctylester, 5-methylbicyclo[2.2.1]hept-2,5-dien-2,3-dicarboxylic acid diisobutylester, 5-methylbicyclo[2.2.1]hept-2,5-dien-2,3-dicarboxylic acid dibutylester, 5-methylbicyclo[2.2.1]hept-2,5-dien-2,3-dicarboxylic acid diisopropylester, 5-methylbicyclo[2.2.1]hept-2,5-dien-2,3-dicarboxylic acid dipropylester, 5-methylbicyclo[2.2.1]hept-2,5-dien-2,3-dicarboxylic acid diethylester, 5-methylbicyclo[2.2.1]hept-2,5-dien-2,3-dicarboxylic acid dimethylester, 6-methylbicyclo[2.2.1]hept-2,5-dien-2,3-dicarboxylic acid ethylhexylester, 6-methylbicyclo[2.2.1]hept-2,5-dien-2,3-dicarboxylic acid dioctylester, 6-methylbicyclo[2.2.1]hept-2,5-dien-2,3-dicarboxylic acid diisobutylester, 6-methylbicyclo[2.2.1]hept-2,5-dien-2,3-dicarboxylic acid dibutylester, 6-methylbicyclo[2.2.1]hept-2,5-dien-2,3-dicarboxylic acid diisopropylester, 6-methylbicyclo[2.2.1]hept-2,5-dien-2,3-dicarboxylic acid dipropylester, 6-methylbicyclo[2.2.1]hept-2,5-dien-2,3-dicarboxylic acid diethylester, 6-methylbicyclo[2.2.1]hept-2,5-dien-2,3-dicarboxylic acid dimethylester, 5,6-dimethylbicyclo[2.2.1]hept-2,5-dien-2,3-dicarboxylic acid ethylhexylester, 5,6-dimethyl bicyclo[2.2.1]hept-2,5-dien-2,3-dicarboxylic acid dioctylester, 5,6-dimethylbicyclo[2.2.1]hept-2,5-dien-2,3-dicarboxylic acid diisobutylester, 5,6-dimethylbicyclo[2.2.1]hept-2,5-dien-2,3-dicarboxylic acid dibutylester, 5,6-dimethylbicyclo[2.2.1]hept-2,5-dien-2,3-dicarboxylic acid diisopropylester, 5,6-dimethylbicyclo[2.2.1]hept-2,5-dien-2,3-dicarboxylic acid dipropylester, 5,6-dimethylbicyclo[2.2.1]hept-2,5-dien-2,3-dicarboxylic acid diethylester, 5,6-dimethylbicyclo[2.2.1]hept-2,5-dien-2,3-dicarboxylic acid dimethylester, and the like.

The transition metal or transition metal precursor according to the present disclosure may be selected from the group consisting of Group 3 to Group 10 in the periodic table, and may preferably be chromium. In the catalyst for ethylene oligomerization according to the present disclosure, the transition metal compound may be a simple inorganic or organic salt, a metal-coordinated complex, or a metallo-organic complex, and may preferably be chromium or a chromium precursor. Preferably, the chromium or chromium precursor may be selected from the group consisting of chromium(III)acetylacetonate, chromium trichloride tristetrahydrofuran, and chromium(III)2-ethylhexanoate.

The catalyst system according to the present disclosure may be produced through a process of producing a ligand coordination complex (catalyst precursor) from the transition metal compound and the R¹—OC(═O)—Y¹—C(═O)OR² or R¹—O—Y²—O—R² backbone structure ligand. A coordination complex produced using the R¹—OC(═O)—Y¹—C(═O)OR² or R¹—O—Y²—O—R² backbone structure ligand and the transition metal compound may be added to a reaction mixture, or the R¹—OC(═O)—Y¹—C(═O)OR² or R¹—O—Y²—O—R² backbone structure ligand and the transition metal compound may be separately added into a reactor, and, thus, a ligand coordination complex with a backbone structure of R¹—OC(═O)—Y¹—C(═O)OR² or R¹—O—Y²—O—R² can be produced. The fact that the ligand coordination complex with a backbone structure of R¹—OC(═O)—Y¹—C(═O)OR² or R¹—O—Y²—O—R² is produced means that the complex is produced in a medium in which a catalytic reaction is conducted. In order to produce the ligand coordination complex, the transition metal compound and the R¹—OC(═O)—Y¹—C(═O)OR² or R¹—O—Y²—O—R² backbone structure ligand are mixed such that a combination ratio of the metal to the ligand is typically about 0.01:1 to 100:1, preferably about 0.1:1 to 10:1, and more preferably 0.5:1-2:1.

The co-catalyst according to the present disclosure may be an arbitrary compound used to produce an active catalyst when it is mixed with the transition metal or transition metal precursor and the R¹—OC(═O)—Y¹—C(═O)OR² or R¹—O—Y²—O—R² backbone structure ligand.

The co-catalyst may be a single compound or a mixture thereof. Examples of the co-catalyst may include organic aluminum compounds, organic boron compounds, organic and inorganic acids, salts, and the like. The organic aluminum compounds may include a compound represented by Chemical Formula AlR₃ (where R is each independently a C₁-C₁₂ alkyl group, an oxygen-containing alkyl group, or a halide) and a compound such as LiAlH₄. Examples thereof may include trimethyl aluminum, triethyl aluminum, triisobutyl aluminum, tri-n-octyl aluminum, methyl aluminum dichloride, methylaluminum dichloride, ethylaluminum dichloride, dimethylaluminum chloride, diethylaluminum chloride, ethylaluminum sesquichloride, methylaluminum sesquichloride, and aluminoxane. Aluminoxane is well known in the art as a typical oligomer compound that can be produced by mixing an alkylaluminum compound, such as trimethylaluminum, with water. Such an oligomer compound may be a linear compound, a cyclic compound, a cage compound, or a mixture thereof. It is believed that commercially available aluminoxanes are generally mixtures of linear and cyclic compounds. Non-limiting examples thereof may include methylaluminoxane, ethylaluminoxane, butylaluminoxane, hexylaluminoxane, octylaluminoxane, decylaluminoxane, or mixtures thereof.

Examples of the organic boron compounds may include boroxine, NaBH4, trimethylboron, triethylboron, dimethylphenylammoniumtetra(phenyl)borate, trityltetra(phenyl)borate, triphenylboron, dimethylphenylammoniumtetra(pentafluorophenyl)borate, sodiumtetrakis[(bis-3,5-trifluoromethyl)phenyl]borate, H⁺(0Et₂)2[(bis-3,5-trifluoromethyl)phenyl]borate, trityltetra(pentafluorophenyl)borate and tris(pentafluorophenyl)boron, trimethylammoniumtetraphenylborate, triethylammoniumtetraphenylborate, tripropylammoniumtetraphenylborate, tributylammoniumtetraphenylborate, trimethylammoniumtetrakis(pentafluorophenyl)borate, triethylammoniumtetrakis(pentafluorophenyl)borate, tripropylammoniumtetrakis(pentafluorophenyl)borate, tributylammonium tetrakis(pentafluorophenyl)borate, aniliniumtetraphenylborate, aniliniumtetrakis(pentafluorophenyl)borate, pyridiniumtetraphenylborate, pyridiniumtetrakis(pentafluorophenyl)borate, ferrocenium tetrakis(pentafluorophenyl)borate, silvertetraphenylborate, silver tetrakis(pentafluorophenyl)borate, tris(pentafluorophenyl)borane, tris(2,3,5,6-tetrafluorophenyl)borane, tris(2,3,4,5-tetraphenylphenyl)borane, tris(3,4,5-trifluorophenyl)borane, and the like.

These organic boron compounds may be used as mixed with the organic aluminum compounds.

The present disclosure provides a catalyst system having a novel structure for providing an ethylene oligomer produced via ethylene oligomerization and a method for producing the same, a catalyst system which can be produced through a simple production process, has an excellent catalytic activity in ethylene oligomerization, and includes the transition metal compound and a method for producing the same, and a method of ethylene oligomerization using the catalyst system.

Further aspects and areas of applicability will become apparent from the description provided herein. It should be understood that various aspects of this disclosure may be implemented individually or in combination with one or more other aspects. It should be understood that the description and specific examples herein are intended for purposes of illustration only and are not intended to limit the scope of the present disclosure.

### DETAILED DESCRIPTION

Example embodiments will now be described more fully.

The illustrative embodiments described in the detailed description and claims are not meant to be limiting. Other embodiments may be utilized, and other changes may be made, without departing from the spirit or scope of the subject matter presented here.

[Method for Producing Ethylene Oligomerization]

The present disclosure provides a method for producing an ethylene oligomer by adding the catalyst for ethylene oligomerization into oligomerization.

In order for the catalyst system of the present disclosure to express a higher catalytic activity when ethylene oligomerization is carried out, it is preferable to use an appropriate reaction solvent and use components, i.e., procatalyst, co-catalyst, and other additives, required for the catalyst system, under the selected reaction conditions with a composition ratio in a predetermined range. Herein, trimerization may be performed in the slurry phase, liquid phase, gas phase, or bulk phase. If the trimerization is performed in the liquid or slurry phase, a reaction solvent may be used as a medium. As a preferable example of the method for producing an ethylene oligomer, the above-described catalyst (for example, procatalyst, co-catalyst) for ethylene oligomer, ethylene, and a solvent may be added into a reactor to react ethylene in ethylene oligomerization, and, thus, an ethylene oligomer can be produced.

In preparing the catalyst used in the present disclosure, the amount of the co-catalyst is in the range of generally 0.1 to 20,000, preferably 1 to 4,000, aluminum or boron atoms per chromium atom. If the concentration of each component is out of the above-described range, the catalytic activity may become too low or an undesirable side reaction such as the production of polymer may occur. In the catalyst system used in the present disclosure, the transition metal or transition metal precursor, the R¹—OC(═O)–Y¹—C(═O)OR² or R¹—O—Y²—O—R² backbone structure ligand and the co-catalyst are added simultaneously or sequentially in arbitrary order into an arbitrary proper solvent in the presence or absence of a monomer, and, thus, an active catalyst can be obtained. For example, the transition metal precursor, the R¹—OC(═O)—Y¹—C(═O)OR² or R¹—O—Y²—O—R² backbone structure ligand, the co-catalyst, and the monomer may be brought into contact with each other simultaneously, or the transition metal precursor, the R¹—OC(═O)—Y¹—C(═O)OR² or R¹—O—Y²—O—R² backbone structure ligand and the co-catalyst may be added simultaneously or sequentially in arbitrary order and then brought into contact with the monomer, or the transition metal precursor and the R¹—OC(═O)—Y¹—C(═O)OR² or R¹—O—Y²—O—R² backbone structure ligand may be added together to form a metal-ligand complex which can be separated and then added to the co-catalyst so as to be brought into contact with the monomer, or the transition metal precursor, the R¹—OC(═O)—Y¹—C(═O)OR² or R¹—O—Y²—O—R² backbone structure ligand and the co-catalyst may be added together to form a metal-ligand complex which can be separated and then brought into contact with the monomer. Examples of a solvent proper for contact between the components of the catalyst or catalyst system may include hydrocarbon solvents, such as heptane, toluene, 1-hexene, and the like, and polar solvents, such as diethyl ether, tetrahydrofuran, acetonitrile, dichloromethane, chloroform, chlorobenzene, methanol, acetone, and the like, but may not be limited thereto.

The reaction conditions for ethylene oligomerization in the presence of the catalyst of the present disclosure are not particularly limited. For example, a reaction temperature may be in the range of 0° C. to 200° C. and preferably 20° C. to 100° C. and a reaction pressure may be in the range of 1 bar to 100 bar and preferably 5 bar to 70 bar. The duration of the reaction may vary depending on the activity of the catalyst system, and a reaction time of 5 minutes to 3 hours may be applied. Thus, the reaction can be completed effectively.

Hereinafter, examples of the present disclosure will be described in more detail. However, the following examples are provided to aid in the understanding of the present disclosure, but shall not be construed as limiting the scope of the present disclosure, and various modifications and changes can be made from the following examples without departing from the spirit of the present disclosure.

[Materials and Analysis Instrument]

The synthesis reactions described below were performed under an inert atmosphere such as nitrogen or argon using Standard Schlenk and Glove Box techniques.

Solvents for synthesis such as tetrahydrofuran (THF), normalhexane (n-Hexane), normalpentane (n-Pentane), diethylether, and methylene chloride (CH₂Cl₂) were passed through an activated alumina column to remove moisture and then used as being preserved on an activated molecular sieve.

Gas chromatography (GC) analysis was performed using an Agilent technologies 7890A GC system under the conditions including a carrier gas of N₂, a carrier gas flow of 2.0 mL/min, a split ratio of 20/l, an initial oven temperature of 50° C., an initial time of 2 min, a ramp of 10° C./min, and a final temperature of 280° C. A column used herein was an HP-5, and ethanol or nonane was quantified to be used as internal standard.

### EXAMPLES

2,2-diisobutyl-1,3-dimethoxypropane and 2-isopropyl-2-isopentyl-1,3-dimethoxypropane (EP 361,493, page 4), 9,9-bis(methoxymethyl)fluorene (EP728,769, page 12), and diethyl-2,3-diisobutylsuccinate (WO 00/63261) were synthesized by the processes disclosed in the corresponding documents, respectively. Methyl aluminoxane, a 10% w/w solution in toluene, was purchased from Albemarle and the other reagents such as trityl(tetrafluorophenyl)borate and triethyl aluminum were purchased from Aldrich chemical company or Strem chemical company unless otherwise noted.

### Example 1

A 300-ml stainless steel reactor was washed with nitrogen in a vacuum and then 50 ml of toluene was added thereto, and 10.0 mmol-Al MAO was added thereto. Then, the temperature was increased to 65° C.

CrCl₃(THF)₃ (0.02 mmol) and 9,9-bis(methoxymethyl)fluorene (0.02 mmol) were introduced into a Schlenk flask, and 10 ml of methylenechloride was added thereto, and the solution was stirred for 4 hours. Then, the solvent was removed under reduced pressure and the resultant solid was suspended in 20 ml of toluene. A 0.01 mmol toluene solution was taken out and then introduced into the reactor.

Ethylene was fed into a pressure reactor under a pressure of 32 bar and then stirred at a stirring speed of 600 rpm. After 30 minutes, the supply of ethylene into the reactor was stopped and the stirring was stopped to stop the reaction. The reactor was cooled to lower than 10° C. After the unreacted ethylene within the reactor was discharged, ethanol mixed with 10 vol % of hydrochloric acid was added to the liquid present in the reactor. A small amount of an organic layer sample was passed through anhydrous magnesium sulfate and dried and then analyzed using GC-FID. The remaining organic layer was filtered to separate a solid wax/polymer product therefrom. After the solid product was dried in an oven at a temperature of 100° C. overnight, a polymer was obtained and the weight of the polymer was checked. The oligomer distribution in the reaction mixture obtained via GC analysis is given in Table 1.

### Example 2

The process of Example 1 was performed in the same manner except that 2,2-diisobutyl-1,3-dimethoxy propane (0.02 mmol) was used instead of 9,9-bis(methoxymethyl)fluorene, and the result of GC analysis and the weight of a polymer are given in Table 1.

### Example 3

The process of Example 1 was performed in the same manner except that 2-isobutyl-2-isopentyl-1,3-dimethoxy propane (0.02 mmol) was used instead of 9,9-bis(methoxymethyl)fluorene, and the result of GC analysis and the weight of a polymer are given in Table 1.

### Comparative Example 1

Zirconium tetrachloride (ZrCl₄) (2.5 mmol) was introduced into a Schlenk flask, and 50 ml of toluene was added thereto with stirring. An ethylaluminum sesquichloride solution (10.0 mmol) was added thereto for 30 minutes. Then, the temperature was increased to 80° C. and a reaction was carried out for 30 minutes. Then, the temperature was lowered to room temperature, and the entire solution was used as a catalyst stock solution. A 0.03 mmol toluene solution was taken out and then introduced into the reactor.

Ethylene was fed into a pressure reactor under a pressure of 32 bar and then stirred at a stirring speed of 600 rpm. After 30 minutes, the supply of ethylene into the reactor was stopped and the stirring was stopped to stop the reaction. The reactor was cooled to lower than 10° C. After the unreacted ethylene within the reactor was discharged, ethanol mixed with 10 vol % of hydrochloric acid was added to the liquid present in the reactor. A small amount of an organic layer sample was passed through anhydrous magnesium sulfate and dried and then analyzed using GC-FID. The remaining organic layer was filtered to separate a solid wax/polymer product therefrom. After the solid product was dried in an oven at a temperature of 100° C. overnight, a polymer was obtained and the weight of the polymer was checked. The oligomer distribution in the reaction mixture obtained via GC analysis is given in Table 1.

### Comparative Example 2

Zirconium tetrachloride (ZrCl₄) (2.5 mmol) was introduced into a Schlenk flask, and 50 ml of toluene was added thereto with stirring. A triethyl aluminum solution (3.9 mmol) was added thereto for 30 minutes with stirring. Then, an ethylaluminum sesquichloride solution (13.6 mmol) was further added thereto for 30 minutes. Then, the temperature was increased to 70° C. and a reaction was carried out for 1 hour. Then, the temperature was lowered to room temperature, and the entire solution was used as a catalyst stock solution. A 0.03 mmol toluene solution was taken out and then introduced into the reactor. Then, oligomerization was carried out in the same manner as in Comparative Example 1. The oligomer distribution in the reaction mixture obtained via GC analysis is given in Table 1.

### Comparative Example 3

Zirconium tetrachloride (ZrCl₄) (0.03 mmol) was introduced into a Schlenk flask, and 5 ml of toluene was added thereto with stirring. N-butanol (0.12 mmol) was added thereto. Then, the temperature was increased to 50° C. and a reaction was carried out for 30 minutes. Then, an ethylaluminum sesquichloride solution (0.12 mmol) was slowly added thereto and stirred for 15 minutes. Then, the temperature was lowered to room temperature, and the entire solution was introduced into the reactor. Then, oligomerization was carried out in the same manner as in Comparative Example 1. The oligomer distribution in the reaction mixture obtained via GC analysis is given in Table 1.

### Comparative Example 4

Tris(2-ethylhexanoate)chromium (Cr(EH)₃) (0.03 mmol) was dissolved in toluene (3 mL) and then, 2,5-dimethylpyrrole (0.09 mmol) was added thereto, and the temperature was lowered to 0° C. Then, a triethylaluminum (0.33 mmol) and diethylaluminum chloride (0.24 mmol) solution was slowly added thereto and a reaction was carried out for 1 hour. The entire solution was introduced into the reactor, and oligomerization was carried out in the same manner as in Comparative Example 1. The oligomer distribution in the reaction mixture obtained via GC analysis is given in Table 1.

### Comparative Example 5

Tris(2-ethylhexanoate)chromium (Cr(EH)₃) (0.03 mmol) was dissolved in toluene (3 mL) and then, 2,5-dimethylpyrrole (0.09 mmol) was added thereto, and the temperature was lowered to 0° C. Then, an ethylaluminum sesquichloride (0.60 mmol) solution was slowly added thereto and a reaction was carried out for 1 hour. The entire solution was introduced into the reactor, and oligomerization was carried out in the same manner as in Comparative Example 1. The oligomer distribution in the reaction mixture obtained via GC analysis is given in Table 1.

### Example 4

The process of Example 1 was performed in the same manner except that diethyl malonate (0.02 mmol) was used instead of 9,9-bis(methoxymethyl)fluorene, and the result of GC analysis and the weight of a polymer are given in Table 1.

### Example 5

The process of Example 1 was performed in the same manner except that diethyl succinate (0.02 mmol) was used instead of 9,9-bis(methoxymethyl)fluorene, and the result of GC analysis and the weight of a polymer are given in Table 1.

### Example 6

The process of Example 1 was performed in the same manner except that diethyl-2,3-diisobutyl-succinate (0.02 mmol) was used instead of 9,9-bis(methoxymethyl)fluorene, and the result of GC analysis and the weight of a polymer are given in Table 1.

### Example 7

The process of Example 1 was performed in the same manner except that diethyl gultarate (0.02 mmol) was used instead of 9,9-bis(methoxymethyl)fluorene, and the result of GC analysis and the weight of a polymer are given in Table 1.

### Example 8

A 300-ml stainless steel reactor was washed with nitrogen in a vacuum and then 40 ml of toluene was added thereto, and 0.2 mmol-Al triethylaluminum was added thereto. Then, the temperature was increased to 65° C.

The catalyst (0.01 mmol) of Example 6 was taken out and introduced into the reactor, and trityltetra(pentafluorophenyl)borate (0.05 mmol) was dissolved in 10 ml of toluene and then introduced into the reactor.

Ethylene was fed into a pressure reactor under a pressure of 32 bar and then stirred at a stirring speed of 600 rpm. After 30 minutes, the supply of ethylene into the reactor was stopped and the stirring was stopped to stop the reaction. The reactor was cooled to lower than 10° C. After the unreacted ethylene within the reactor was discharged, ethanol mixed with 10 vol % of hydrochloric acid was added to the liquid present in the reactor. A small amount of an organic layer sample was passed through anhydrous magnesium sulfate and dried and then analyzed using GC-FID. The remaining organic layer was filtered to separate a solid wax/polymer product therefrom. After the solid product was dried in an oven at a temperature of 100° C. overnight, a polymer was obtained and the weight of the polymer was checked. The oligomer distribution in the reaction mixture obtained via GC analysis is given in Table 1.

### Example 9

The process of Example 7 was performed in the same manner except that trimethyl aluminum (0.2 mmol) was used instead of triethylaluminum, and the result of GC analysis and the weight of a polymer are given in Table 1.

TABLE 1
Result of oligomerization

| | | | | | Comparative | Comparative | Comparative | Comparative | Comparative |
|---|---|---|---|---|---|---|---|---|---|
| | | Example 1 | Example 2 | Example 3 | Example 1 | Example 2 | Example 3 | Example 4 | Example 5 |
| Activity | | 62.5 | 47.2 | 45 | 6.7 | 13.7 | 1.7 | 1.52 | 1.47 |
| (Kg-Olefins/ | | | | | | | | | |
| g-[M]/hr) | | | | | | | | | |
| Oligomer | C4″ | 3.6 | 3.8 | 3.9 | 29.8 | 30.7 | 18.9 | 0.7 | 0.7 |
| Distribution | (wt %) | | | | | | | | |
| | C6″ | 18.3 | 18.1 | 18.4 | 20.8 | 21.0 | 14.5 | 90.2 | 91.2 |
| | (wt %) | | | | | | | | |
| | C8″ | 22.1 | 22.3 | 22.0 | 10.2 | 9.5 | 10.2 | 0.3 | 0.3 |
| | (wt %) | | | | | | | | |
| | C10″- | 40.2 | 39.8 | 39.6 | 28.7 | 29.3 | 31.3 | 4.6 | 2.6 |
| | C18″ | | | | | | | | |
| | (wt %) | | | | | | | | |
| | C20″+ | 12.0 | 12.3 | 12.7 | 6.6 | 7.3 | 14.3 | — | — |
| | (wt %) | | | | | | | | |
| Polymer | (wt %) | 3.5 | 3.3 | 3.1 | 3.6 | 2.1 | 10.5 | 3.8 | 5.1 |

| | | Example 4 | Example 5 | Example 6 | Example 7 | Example 8 | Example 9 |
|---|---|---|---|---|---|---|---|
| Activity | | 21.1 | 71.2 | 166.7 | 82.3 | 128.6 | 118.8 |
| (Kg-Olefins/ | | | | | | | |
| g-[M]/hr) | | | | | | | |
| Oligomer | C4″ | 4.5 | 3.7 | 3.5 | 3.3 | 3.5 | 3.2 |
| Distribution | (wt %) | | | | | | |
| | C6″ | 19.2 | 19.7 | 18.6 | 19.6 | 18.4 | 18.9 |
| | (wt %) | | | | | | |
| | C8″ | 20.3 | 21.0 | 21.8 | 20.9 | 21.6 | 22.1 |
| | (wt %) | | | | | | |
| | C10″- | 38.9 | 39.7 | 41.7 | 38.9 | 41.5 | 40.3 |
| | C18″ | | | | | | |
| | (wt %) | | | | | | |
| | C20″+ | 14.2 | 13.4 | 12.1 | 14.5 | 12.3 | 13.3 |
| | (wt %) | | | | | | |
| Polymer | (wt %) | 2.7 | 2.3 | 2.1 | 2.3 | 2.4 | 1.9 |

As listed in Table 1, it can be seen from the results of oligomerization according to Example 1 to Example 9 of the present disclosure that the novel oligomerization catalyst system of the present disclosure has a high catalytic activity. Further, in the oligomer distribution, C8″ (C8 olefin) and C10-C18 olefins are highly distributed. Thus, it can be seen that the novel oligomerization catalyst system of the present disclosure is very useful for C8-C18 α-olefins.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A catalyst system for ethylene oligomerization comprising:
a transition metal or transition metal precursor;
a ligand with a backbone structure expressed by the following Chemical Formula 1 or Chemical Formula 2; and
a co-catalyst:
R¹—OC(═O)—Y¹—C(═O)OR² [Chemical Formula 1]
(wherein R¹, R² are each independently hydrocarbyl, substituted hydrocarbyl, heterohydrocarbyl, or substituted heterohydrocarbyl, and Y¹ represents a group connecting CO(═O)), and
R¹—O—Y²—O—R² [Chemical Formula 2]
(wherein R¹, R² are each independently hydrocarbyl, substituted hydrocarbyl, heterohydrocarbyl, or substituted heterohydrocarbyl, and Y² represents a group connecting O and is a linear, branched, or cyclic alkyl group having 3 or more carbon atoms, or hetero hydrocarbyl, or substituted heterohydrocarbyl).

2. The catalyst system for ethylene oligomerization of claim 1, wherein the ligand with a backbone structure expressed by Chemical Formula 1 or Chemical Formula 2 is a diether compound represented by the following General Formula 1,
R¹R²C(CH₂OR³)(CH₂OR⁴) (1)
(wherein R¹ and R² are identical or different and represent C 1-C18 alkyl groups, C3-C18 cycloalkyl groups, or C7-C18 aryl radical groups; and R³ and R⁴ are identical or different and represent C1-C4 alkyl radical groups or cyclic or polycyclic groups in which the carbon atom at position 2 contains 2 or 3 unsaturated bonds and which have 5, 6, or 7 carbon atoms), or
any one selected from dicarboxylic acid ester compounds represented by the following General Formula 2 to General Formula 6: 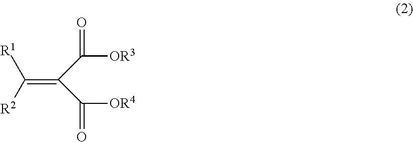
(wherein R¹ and R² are each independently hydrogen or a linear or branched alkyl group having 1 to 20 carbon atoms, a cyclic alkyl group or alkenyl group having 3 to 20 carbon atoms, an aryl group having 6 to 20 carbon atoms, or an arylalkyl group or alkyl aryl group having 7 to 20 carbon atoms and are combined to form a cycle, and R³ and R⁴ are each independently a linear or branched alkyl group having 1 to 20 carbon atoms), 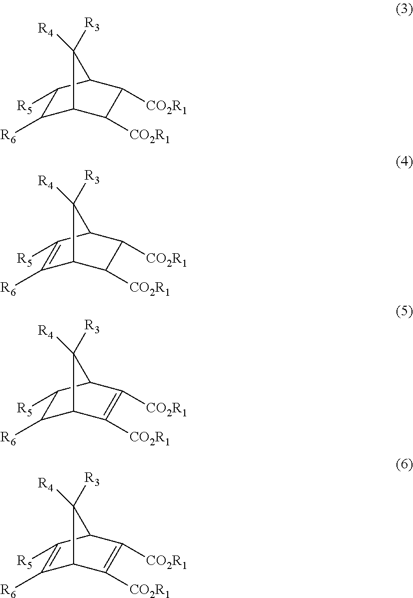
(wherein R₁ and R₂ are identical to or different from each other and represent linear, branched, or cyclic alkyl groups or alkenyl groups having 1 to 20 carbon atoms, aryl groups having 6 to 20 carbon atoms, or arylalkyl groups or alkylaryl groups having 7 to 20 carbon atoms; and R₃, R₄, R₅ and R₆ are identical to or different from each other and represent hydrogen, linear, branched, or cyclic alkyl groups or alkenyl groups having 1 to 20 carbon atoms, aryl groups having 6 to 20 carbon atoms, or arylalkyl groups or alkylaryl groups having 7 to 20 carbon atoms).

3. The catalyst system for ethylene oligomerization of claim 2, wherein the dicarboxylic acid ester of General Formula 2 is any one selected from malonate, succinate, glutarate, pivalate, adipate, sebacate, malate, naphthalene dicarboxylate, trimellitate, benzene-1,2,3-tricarboxylate, pyromellitate, and carbonate.

4. The catalyst system for ethylene oligomerization of claim 3, wherein the dicarboxylic acid ester of General Formula 2 is any one selected from diethyl malonate, dibutyl malonate, dimethyl succinate, diethyl succinate, dinormalpropyl succinate, diisopropyl succinate, 1,1-dimethyl-dimethylsuccinate, 1,1-dimethyl-diethylsuccinate, 1,1-dimethyl-dinormalpropylsuccinate, 1,1-dimethyl-diisopropylsuccinate, 1,2-dimethyl-dimethylsuccinate, 1,2-dimethyl-diethylsuccinate, ethyl-dimethylsuccinate, ethyl-diethylsuccinate, ethyl-dinormalpropylsuccinate, ethyl-diisopropylsuccinate, 1,1-diethyl-dimethylsuccinate, 1,1-diethyl-diethylsuccinate, 1,1-diethyl-dimethylsuccinate, 1,2-diethyl-dimethylsuccinate, 1,2-diethyl-diethylsuccinate, 1,2-diethyl-dinormalpropylsuccinate, 1,2-diethyl-diisopropylsuccinate, normalpropyl-dimethylsuccinate, normalpropyl-diethylsuccinate, normalpropyl-dinormalpropylsuccinate, normalpropyl-diisopropylsuccinate, isopropyl-dimethylsuccinate, isopropyl-diethylsuccinate, isopropyl-dinormalpropylsuccinate, isopropyl-diisopropylsuccinate, 1,2-diisopropyl-dimethylsuccinate, 1,2-diisopropyl-diethylsuccinate, 1,2-diisopropyl-dinormalpropylsuccinate, 1,2-diisopropyl-diisopropylsuccinate, normalbutyl-dimethylsuccinate, normalbutyl-diethylsuccinate, normalbutyl-dinormalpropylsuccinate, normalbutyl-diisopropylsuccinate, isobutyl-dimethyl succinate, isobutyl-diethylsuccinate, isobutyl-dinormalpropylsuccinate, isobutyl-diisopropylsuccinate, 1,2-dinormalbutyl-dimethylsuccinate, 1,2-dinormalbutyl-diethylsuccinate, 1,2-dinormalbutyl-dinormalpropylsuccinate, 1,2-dinormalbutyl-diisopropylsuccinate, 1,2-dinormalbutyl-dimethylsuccinate, 1,2-diisobutyl-dimethylsuccinate, 1,2-diisobutyl-diethylsuccinate, 1,2-diisobutyl-dinormalpropylsuccinate, 1,2-diisobutyl-diisopropylsuccinate, diethyl adipate, dibutyl adipate, diethyl sebacate, dibutyl sebacate, diethyl malate, di-n-butyl malate, diethyl naphthalene dicarboxylate, dibutyl naphthalene dicarboxylate, triethyl trimellitate, tributyl trimellitate, triethyl benzene-1,2,3-tricarboxylate, tributyl benzene-1,2,3-tricarboxylate, tetraethyl pyromellitate, and tetrabutyl pyromellitate.

5. The catalyst system for ethylene oligomerization of claim 1, wherein the transition metal or transition metal precursor is chromium or a chromium precursor.

6. The catalyst system for ethylene oligomerization of claim 5, wherein the chromium or chromium precursor is selected from the group consisting of chromium(III)acetylacetonate, chromium trichloride tristetrahydrofuran, and chromium(III)2-ethylhexanoate.

7. The catalyst system for ethylene oligomerization of claim 1, wherein the co-catalyst is methylaluminoxane, ethylaluminoxane, butylaluminoxane, hexylaluminoxane, octylaluminoxane, decylaluminoxane, or a mixture thereof.

8. The catalyst system for ethylene oligomerization of claim 1, wherein the co-catalyst is a combination of trialkylaluminum and the following borate or boron compound:
dimethylphenylammoniumtetra(phenyl)borate, trityltetra(phenyl)borate, triphenylboron, dimethylphenylammoniumtetra(pentafluorophenyl)borate, sodiumtetrakis[(bis-3,5-trifluoromethyl)phenyl]borate, H⁺(0Et₂)2[(bis-3,5-trifluoromethyl)phenyl]borate, trityltetra(pentafluorophenyl)borate and tris(pentafluorophenyl)boron, trimethylammoniumtetraphenylborate, triethylammoniumtetraphenylborate, tripropylammoniumtetraphenylborate, tributylammoniumtetraphenylborate, trimethylammoniumtetrakis(pentafluorophenyl)borate, triethylammoniumtetrakis(pentafluorophenyl)borate, tripropylammoniumtetrakis(pentafluorophenyl)borate, tributylammonium tetrakis(pentafluorophenyl)borate, aniliniumtetraphenylborate, aniliniumtetrakis(pentafluorophenyl)borate, pyridiniumtetraphenylborate, pyridiniumtetrakis(pentafluorophenyl)borate, ferrocenium tetrakis(pentafluorophenyl)borate, silvertetraphenylborate, silver tetrakis(pentafluorophenyl)borate, tris(pentafluorophenyl)borane, tris(2,3,5,6-tetrafluorophenyl)borane, tris(2,3,4,5-tetraphenylphenyl)borane, and tris(3,4,5-trifluorophenyl)borane.

9. A method for producing ethylene oligomerization by bringing an ethylene monomer into contact with a catalyst system for ethylene oligomerization including a transition metal or transition metal precursor, a ligand with a backbone structure expressed by the following Chemical Formula 1 or Chemical Formula 2, and a co-catalyst:
R¹R²C(CH₂OR³)(CH₂OR⁴) (1)
(wherein R¹, R² are each independently hydrocarbyl, substituted hydrocarbyl, heterohydrocarbyl, or substituted heterohydrocarbyl, and Y¹ represents a group connecting CO(═O)), and
R¹—O—Y²—O—R² [Chemical Formula 2]
(wherein R¹, R² are each independently hydrocarbyl, substituted hydrocarbyl, heterohydrocarbyl, or substituted heterohydrocarbyl, and Y² represents a group connecting O and is a linear, branched, or cyclic alkyl group having 3 or more carbon atoms, or hetero hydrocarbyl, or substituted heterohydrocarbyl).

10. The catalyst system for ethylene oligomerization of claim 2, wherein the transition metal or transition metal precursor is chromium or a chromium precursor.

11. The catalyst system for ethylene oligomerization of claim 2, wherein the co-catalyst is methylaluminoxane, ethylaluminoxane, butylaluminoxane, hexylaluminoxane, octylaluminoxane, decylaluminoxane, or a mixture thereof.

12. The catalyst system for ethylene oligomerization of claim 2, wherein the co-catalyst is a combination of trialkylaluminum and the following borate or boron compound:
dimethylphenylammoniumtetra(phenyl)borate, trityltetra(phenyl)borate, triphenylboron, dimethylphenylammoniumtetra(pentafluorophenyl)borate, sodiumtetrakis [(bis-3,5-trifluoromethyl)phenyl]borate, H⁺(0Et₂)2[(bis-3,5-trifluoromethyl)phenyl]borate, trityltetra(pentafluorophenyl)borate and tris(pentafluorophenyl)boron, trimethylammoniumtetraphenylborate, triethylammoniumtetraphenylborate, tripropylammoniumtetraphenylborate, tributylammoniumtetraphenylborate, trimethylammoniumtetrakis(pentafluorophenyl)borate, triethylammoniumtetrakis(pentafluorophenyl)borate, tripropylammoniumtetrakis(pentafluorophenyl)borate, tributylammonium tetrakis(pentafluorophenyl)borate, aniliniumtetraphenylborate, aniliniumtetrakis(pentafluorophenyl)borate, pyridiniumtetraphenylborate, pyridiniumtetrakis(pentafluorophenyl)borate, ferrocenium tetrakis(pentafluorophenyl)borate, silvertetraphenylborate, silver tetrakis(pentafluorophenyl)borate, tris(pentafluorophenyl)borane, tris(2,3,5,6-tetrafluorophenyl)borane, tris(2,3,4,5-tetraphenylphenyl)borane, and tris(3,4,5-trifluorophenyl)borane.
